(19) United States
(12) Patent Application Publication
(10) Pub. No.: US 2021/0085765 A1
(43) Pub. Date: Mar. 25, 2021

(54) METHODS AND COMPOSITIONS FOR PRE-EMPTIVE TREATMENT OF GRAFT VERSUS HOST DISEASE

(71) Applicant: KAMADA LTD., Rehovot (IL)

(72) Inventors: Naveh TOV, Haifa (IL); Michal STEIN, Hod Hasharon (IL)

(73) Assignee: KAMADA LTD., Rehovot (IL)

(21) Appl. No.: 17/109,551

(22) Filed: Dec. 2, 2020

Related U.S. Application Data
(63) Continuation-in-part of application No. 16/918,121, filed on Jul. 1, 2020.
Application No. 16/918,121 is a continuation-in-part of application No. PCT/IL2018/051415, filed on Dec. 31, 2018.
(60) Provisional application No. 62/612,635, filed on Jan. 1, 2018.

(51) Int. Cl. A61K 38/57; A61K 9/00; A61P 37/06

(52) U.S. Cl. CPC A61K 38/57; A61P 37/06; A61K 9/0078

(57) ABSTRACT

Methods for pre-emptive prophylactic treatment of graft versus host disease (GvHD) are disclosed. Such methods comprise the administration of Alpha-1 Antitrypsin (AAT) for pre-emption of GvHD.

### FIELD OF THE INVENTION

The present invention relates to methods for pre-emptive treatment of graft versus host disease (GvHD). The method of the invention comprises the administration of a multiple variable dose regimen of Alpha-1 Antitrypsin (AAT) for pre-emption of acute GvHD.

### BACKGROUND

Acute GvHD

Hematopoietic cellular transplantation (HCT or BMT) is an important treatment for high-risk hematologic malignancies whose curative potential depends on the graft-versus-leukemia (GVL) effect. Graft-versus-host disease (GvHD), the major cause of non-relapse mortality (NRM) after HCT, is closely associated with GVL. Pre-transplant clinical risk factors for GvHD include the degree of human leukocyte antigen (HLA) match between donor and recipient, recipient age, donor type, and the intensity of the conditioning regimen. Some centers use one or more of these risk factors to guide GvHD prophylaxis, such as the use of anti-thymocyte globulin (ATG, thymoglobulin) when the donor is not an HLA-identical sibling, but such approaches are globally immunosuppressive and carry their own risks, in particular of opportunistic infections.

Acute GvHD (aGvHD) affects 40% to 60% of patients and targets the skin, liver, and gastrointestinal (GI) tract. The median onset of acute GvHD is approximately 1 month after transplant. The initial treatment for GvHD typically involves high doses of systemic corticosteroids, but up to 50% of patients do not respond to this approach. These patients may develop steroid refractory (SR) GvHD, which most often involves the GI tract. This is the primary driver of lethal GvHD, and by extension, non-relapse mortality after HCT. However, until now there has been no reliable method to identify the patients at high risk for SR GvHD before GvHD actually develops.

Chronic GvHD

Historically, acute GVHD was defined as any reaction occurring within the first 100 days after transplant, and chronic GVHD (cGVHD) was defined as reactions occurring after 100 days. However, this definition oversimplifies the differences in the presentation of the reactions. Nowadays, the definition is based on differences in the presentation of the associated reactions, though there may be overlap(s) between the two. Accordingly, some patients may present with chronic GVHD earlier than day 100 post transplantation, and conversely some might present acute GVHD after day 100.

The clinical manifestations of cGVHD may be restricted to a single organ or can be disseminated, with profound impact on quality of life. Fibrosis affecting skin and internal organs is the predominant clinical feature of cGVHD. The clinical manifestations usually appear within the first two years after transplantation.

The pathophysiology of cGVHD involves inflammation, cellular and humoral immunity activation and fibrosis. In addition, organ scoring of cGvHD are usually severe (stages 3/4 or 4).

The immunological complication in cGVHD resembles autoimmune diseases with clinical manifestations of collagen vascular diseases, such as oral lichen planus, keratoconjunctivitis sicca, xerostomia, polyserositis, esophagitis and esophageal stricture, vaginal ulceration and stenosis, intrahepatic obstructive liver disease, obstructive pulmonary disease, scleroderma, fasciitis and myositis.

GvHD Biomarker Algorithms Predict HCT Outcomes

The concentrations of several protein biomarkers (e.g., ST2, REG3α, IL2Rα, TNFR1, hepatocyte growth factor, and elafin) in the serum are increased in patients with GvHD. Combinations of the levels of ST2 and REG3α, with or without TNFR1, have now been proven to be prognostic for GvHD outcomes when measured at the time GvHD is diagnosed. Recently, serum samples obtained from patients participating in the Mount Sinai Acute GvHD International Consortium (MAGIC) were used to develop and validate an algorithm that uses the combination of ST2 and REG3a concentrations, measured on Day 7 post-HCT, to predict the development of non-relapse mortality (NRM). Samples from Day 7 post-HCT were used to develop the algorithm because GvHD rarely develops in the first week after HCT, providing sufficient time to test pre-emptive treatment strategies. The MAGIC algorithm identified a high risk (HR) group in the training set whose NRM (28%) was significantly greater (p<0.001) than that of the low risk (LR) group (7%). Application of this algorithm to the test set produced similar, highly statistically significant differences between HR and LR groups. A second validation was performed in the multicenter set and again detected large differences between the groups, with an HR 6-month NRM of 26% versus 10% in the LR group (p<0.001). The proportion of patients in the HR group was similar in all 3 patient sets (16% to 20%) and since the relapse rates were consistently equivalent in both risk groups the HR patients experienced significantly worse overall survival (p<0.001). Importantly, GvHD was the driver of NRM. HR patients were 3 times more likely to die from GvHD than LR patients (HR 19% vs. LR 6%, p<0.001), a finding explained by the much higher 6-month incidence of SR GvHD in high risk patients compared to LR patients (35% vs 15%, p<0.001). It is likely that the blood biomarker concentrations on Day 7 reflect subclinical GI pathology, a notion that is reinforced by the fact that ST2 and REG3α, the two biomarkers that performed the best in the models, are closely associated with GI GvHD.

Several pre-HCT clinical risk factors predict a higher risk of NRM, such as HLA mismatch, non-family member donors, age of the recipient, and the intensity of the conditioning regimen. Importantly, the MAGIC algorithm also stratified patients into distinct risk groups independently of patient age [≤21 y: HR 27% vs LR 6%, p<0.001; >21 y: HR 29% vs LR 8%, p<0.001], conditioning regimen [reduced intensity: HR 37% vs LR 8%, p<0.001; full intensity: HR 25% vs LR 8%, p<0.001] or the use of thymoglobulin in the conditioning regimen [ATG given: HR 31% vs LR 8%, p<0.001; no ATG: HR 28% vs LR 8%, p<0.001]. Relapse rates were equivalent within all the clinical risk factor subgroups, resulting in a decrease of at least 20% in overall survival for HR patients

Recently an attempt to increase the sensitivity of the MAGIC algorithm was made by re-testing low risk patients after a further week (Day 14), with the reasoning that the concentration of GvHD biomarkers might be higher one week closer to the expected onset of GvHD. Serum samples from both Day 7 and Day 14 were available for 768 MAGIC patients. The levels of ST2 and REG3a were measured on Day 7 in all patients and 145 (19%) and 623 (81%) were identified as high and low risk respectively, with distinctly different predictions for NRM (26% vs 8%, p<0.001). Twenty low risk patients (3%) developed GvHD between Day 7 and Day 14 and were excluded from further analysis. When the test was repeated on Day 14 in the remaining 603 LR patients, 60 additional HR patients were identified whose NRM risk was similar to the day 7 HR patients and significantly higher than the 543 patients who remained LR (24% vs 6%, p<0.001). Compared to the Day 7 test alone, repeat testing on Day 14 resulted in a large improvement in sensitivity for NRM prediction (46% to 61%) with only a modest loss of specificity (80% to 75%). Serial testing at both Days 7 and 14 significantly increases the proportion of patients potentially eligible to participate in a GvHD pre-emptive treatment trial from 19% (145/768) of the total population to 27% (205/768).

Plasma derived AAT (pAAT) is currently used therapeutically for the treatment of pulmonary emphysema in patients who have a genetic AAT deficiency, also known as Alpha-1 Antitrypsin Deficiency or Congenital Emphysema. Purified pAAT has been approved for replacement therapy (also known as “augmentation therapy”) in these patients. There is a continuous effort targeted at producing recombinant AAT, but as of today there is no approved recombinant product. The endogenous role of AAT in the lungs is predominantly to regulate the activity of neutrophil elastase, which breaks down foreign proteins present in the lung. In the absence of sufficient quantities of AAT, the elastase breaks down lung tissue, which over time results in chronic lung tissue damage and emphysema.

Several clinical trials have addressed the potential benefit of AAT therapy to individuals with normal AAT production (i.e. not defined as AAT deficient subjects). Indications explored have included islet and lung transplantation, TDM, graft-versus-host disease, acute myocardial infarction, and cystic fibrosis. The initial dosing plan in many of these trails was taken from the long-standing protocols developed for AAT augmentation therapy in AAT-deficient patients.

However, the timing, dosage and duration of AAT treatment required for the pre-emption therapy of GvHD cannot be simply extrapolated from those found to be effective in treating the genetic AAT deficiency and the disorders associated thereto. There is therefore an unmet need for the development of an effective prevention and pre-emptive treatment of GvHD.

### SUMMARY

The present invention provides methods for pre-emptive treatment of GvHD, by employing a multiple variable dose regimen of AAT administration.

The present invention is based in part on AAT early intervention driven by biomarkers. An algorithm is used to identify patients at risk for non-relapse mortality on day 7-14 following bone marrow transplantation (BMT). The algorithm utilizes biomarkers for prediction of mortality risk. Non-relapse mortality is closely related to non-responsiveness to steroids, which are the current standard of care treatment for GvHD. AAT early intervention, based on risk prediction and prior to the development of the clinical symptoms of GvHD, can protect patients from GvHD occurrence or further disease deterioration, such as steroid refractory acute GVHD.

Surprisingly, it has been found that AAT reduces non-relapse mortality (NRM) in patients identified as being at high risk of developing steroid resistant GvHD (SR GvHD). Moreover, AAT prolonged the survival of high-risk patients, compared to matched control patients at high risk who were not treated with AAT. It is known that about 30%-50% of patients undergoing allogeneic hematopoietic transplantation are prone (have a high risk) to develop GvHD and moreover SR GvHD. Hence, as disclosed herein, AAT is capable of improving the course of GvHD, increasing the survival rate of high-risk patients and reducing the percentage of patients developing severe organ conditions. According to some embodiments, the is provided a method for reducing the risk to develop graft versus host disease (GvHD) in a subject in need thereof, the method comprising administering to the subject a pharmaceutical composition comprising alpha-1 antitrypsin (AAT).

According to some embodiments, said administering is performed post transplantation. According to some embodiments, said transplantation is hematopoietic cellular transplantation. According to some embodiments, said transplantation is bone marrow transplantation. According to some embodiments, said administering is performed at least a week post transplantation.

According to some embodiments, the pharmaceutical composition is administered in combination with at least one additional pharmaceutically active agent.

According to some embodiments, the at least one additional pharmaceutically active agent is selected from the group of: anti-inflammatory agents, immunomodulatory agents, immunosuppressive agents, Janus-associated kinase inhibitors, methotrexate, mTOR inhibitors, proteasome inhibitors, extracorporeal photopheresis, anti-inflammatory monoclonal antibodies, cytotoxic monoclonal antibodies, mesenchymal stem cell-based therapies and cannabis-based therapies.

According to some embodiments, the subject in need thereof is a subject having a high risk of developing GvHD.

According to some embodiments, said GvHD is steroid refractory GvHD.

According to some embodiments, said reducing the risk to develop GvHD comprises preventing the development of GvHD.

According to some embodiments, said reducing the risk to develop GvHD comprises increasing survival.

According to some embodiments, said reducing the risk to develop GvHD comprises reducing the cumulative incidence of non-relapse mortality.

According to some embodiments, said reducing the risk to develop GvHD comprises preventing or reducing the severity of GvHD effects on organs.

According to some embodiments, said reducing the risk to develop GvHD comprises reducing relapse. It is to be understood that relapse refers to the disease, treatment of which required the transplantation, where GvHD is a severe side effect of said treatment (namely, of the transplantation). The disease may be cancer, such as, lymphoma, kidney failure, heart failure, or any disease requiring organ transplantation.

According to some embodiments, said reducing the risk to develop GvHD comprises inducing at least partial response. According to some embodiments, said reducing the risk to develop GvHD comprises inducing complete response.

According to some embodiments, said administering the pharmaceutical composition comprises administering said pharmaceutical composition in a multiple dosage regimen.

According to some embodiments, said administering comprises intravenous administration. According to some embodiments, said administering comprises oral administration, or administration via inhalation.

According to some embodiments, said GvHD is acute GvHD. According to some embodiments, said GvHD is chronic GvHD.

According to one aspect, the present invention provides a method for the pre-emptive treatment of acute GvHD in a subject in need thereof, the method comprising administering to the subject AAT in a multiple variable dosage regimen sufficient to prevent or reduce the severity of GvHD in the subject. According to certain embodiments, the subject has recently undergone HCT.

According to another aspect, the present invention provides a use of the composition comprising alpha 1-antitrypsin (AAT) or functional variant thereof in the manufacture of a medicament for the pre-emptive treatment of GvHD in a subject in need thereof.

According to certain embodiments, the subject is at high risk for the development of steroid-refractory GvHD. According to certain embodiments, the subject was identified as being at high risk for the development of steroid-refractory GvHD by measuring level(s) of at least one of ST2 and Reg3α in a blood sample collected from the subject.

According to certain embodiments, the multiple variable dosage regimen comprises administering AAT at a total cumulative dose selected from the group consisting of about 540, 700, 765, 960, 1000, 1,440, 1500, and 3000 mg/KgBW.

According to certain embodiments, the multiple variable dosage regimen comprises about 16 administrations up to the total cumulative dose. According to certain embodiments, the multiple variable dosage regimen length is from about 4 to about 10 weeks. According to certain embodiments, each portion dose comprises from about 30 mg AAT/KgBW to about 180 mg AAT/KgBW. According to certain embodiments, each portion dose comprises 30, 45, 90, 120, 180, or 240 mg AAT/KgBW. According to certain embodiments, the multiple portion doses are administered at intervals of from about 2-4 days to about 1-4 weeks. According to certain embodiments, the intervals are selected from constant intervals and variable intervals. According to certain embodiments, the multiple portion doses contain the same amount of AAT. According to certain embodiments, the multiple portion doses contain variable amounts of AAT. According to certain embodiments, the multiple portion doses are administered at intervals of about 3 days. According to certain embodiments, the amount of AAT decreases from the first dose administered to the second dose administered. According to certain embodiments, the GvHD is steroid refractory GvHD. According to certain embodiments, the steroid-refractory GvHD is resistant to the effects of glucocorticoids. According to certain embodiments, the AAT is selected from the group consisting of plasma-derived AAT and recombinant AAT. According to certain embodiments, the AAT is administered within a pharmaceutical composition. According to certain embodiments, the AAT is administered intravenously. According to certain embodiments, the AAT is administered by oral administration or via inhalation.

According to another aspect, the present invention provides a method for treating or preventing GvHD comprising the administration of AAT to a subject in need thereof at an initial dose of about 70 mg/kg to about 95 mg/kg on day 1 of the administration period followed by 30 mg/kg to about 50 mg/kg during a multiple dosing period. According to certain embodiments, the GvHD is severe or lethal GvHD.

According to certain embodiments, the initial dose is about 90 mg/kg. According to certain embodiments, about 45 mg/kg of AAT is administered during the multiple dosing period.

According to certain embodiments, the dose of about 45 mg/kg of AAT is administered about twice weekly. According to certain embodiments, the AAT is administered for about 8 weeks.

According to certain embodiments, the administration commences within 7 to 16 days after bone marrow transplantation.

Any route of administration as is known in the art to be suitable for AAT administration can be used according to the teachings of the present invention. According to certain exemplary embodiments, the AAT is administered intravenously (i.v.). According to some embodiments, the AAT is administered by oral administration. According to some embodiments, the AAT is administered via inhalation. According to other embodiments, the AAT is administered by subcutaneous administration. The AAT is typically administered within a pharmaceutical composition formulated to complement the route of administration.

According to some embodiments, the method further comprises administration of one or more of an anti-inflammatory agent, an immunosuppressive agent, an immunomodulatory agent, an anti-microbial agent, or a combination thereof.

Other objects, features and advantages of the present invention will become clear from the following description.

### BRIEF DESCRIPTION OF THE DRAWINGS

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

In the Figures:

FIG. 1 represents the screening and enrollment of the patients in the trial.

FIG. 2A represents interim NRM data (n=30).

FIG. 2B represents interim overall survival data (n=30).

FIG. 2C represents interim percentage of patients (n=26) with stage 4 (severe) skin GvHD.

FIG. 2D represents interim percentage of patients (n=26) with stage 3/4 (severe) lower gastrointestinal (LGI) tract at stage 3/4 (severe).

FIG. 3A exhibits six-month cumulative incidence of non-relapse mortality in AAT treated patients (10%) and controls (16%).

FIG. 3B exhibits six-month cumulative incidence of disease relapse in AAT treated patients (7%) and controls (18%).

FIG. 3C exhibits six-month overall survival in AAT treated patients (86%) and controls (78%).

FIG. 4A exhibits median MAGIC algorithm probability (MAP) at study entry (day 7 or 14 post-HCT) in AAT treated patients (0.22, n=30) and controls (0.23, n=90).

FIG. 4B exhibits median MAP at day 28 post-HCT in AAT treated patients (0.10, n=30) and controls (0.14, n=82). Box and whisker plots show median MAP with whiskers extending from the 10th to the 90th percentile. Values outside this range are drawn as individual points.

### DETAILED DESCRIPTION OF THE INVENTION

The present invention discloses a multiple-variable AAT dosage method for pre emption or prevention of acute GvHD.

### Definitions

As used herein, “a” or “an” may mean one or more than one of an item.

As used herein the term “about” refers to the designated value±10%.

As used herein, the term “Alpha-1 Antitrypsin” (AAT) refers to a glycoprotein that in nature is produced by the liver and lung epithelial cells and is secreted into the circulatory system. AAT belongs to the Serine Proteinase Inhibitor (Serpin) family of proteolytic inhibitors. This glycoprotein consists of a single polypeptide chain containing one cysteine residue and 12-13% of the total molecular weight of carbohydrates. AAT has three N-glycosylation sites, at asparagine residues 46, 83, and 247, which are occupied by mixtures of complex bi- and triantennary glycans. This gives rise to multiple AAT isoforms, having isoelectric points in the range of 4.0 to 5.0. The glycan monosaccharides include N-acetylglucosamine, mannose, galactose, fucose, and sialic acid. AAT serves as a pseudo-substrate for elastase; elastase attacks the reactive center loop of the AAT molecule by cleaving the bond between methionine358-serine359 residues to form an AAT-elastase complex. This complex is rapidly removed from the blood circulation. AAT is also referred to as “alpha-1 Proteinase Inhibitor” (API). The term “glycoprotein” as used herein refers to a protein or peptide covalently linked to a carbohydrate. The carbohydrate may be monomeric or composed of oligosaccharides. It is to be explicitly understood that any AAT as is or will be known in the art, including plasma-derived AAT and recombinant AAT can be used according to the teachings of the present invention.

As used herein “analog of alpha-1-antitrypsin” may mean a compound having alpha-1-antitrypsin-like activity. In one embodiment, an analog of alpha-1-antitrypsin is a functional derivative of alpha-1-antitrypsin. Ina particular embodiment, an analog of alpha-1-antitrypsin is a compound capable of significantly reducing serine protease activity. For example, an inhibitor of serine protease activity has the capability of inhibiting the proteolytic activity of trypsin, elastase, kallikrein, thrombin, cathepsin G, chymotrypsin, plasminogen activators, plasmin, and/or other serine proteases.

“Recombinant AAT” as used herein, refers to AAT that is the product of recombinant DNA or transgenic technology. The phrase, “recombinant AAT,” also includes functional fragments of AAT, chimeric proteins comprising AAT, or functional fragments thereof, fusion proteins or fragments of AAT, homologues obtained by analogous substitution of one or more amino acids of AAT, and species homologues. For example, the gene coding for AAT can be inserted into a mammalian gene encoding a milk whey protein in such a way that the DNA sequence is expressed in the mammary gland as described in, e.g., U.S. Pat. No. 5,322,775, which is herein incorporated by reference for its teaching of a method of producing a proteinaceous compound. “Recombinant AAT,” also refers to AAT proteins synthesized chemically by methods known in the art such as, e.g., solid-phase peptide synthesis. Amino acid and nucleotide sequences for AAT and/or production of recombinant AAT are described by, e.g., U.S. Pat. Nos. 4,711,848; 4,732,973; 4,931,373; 5,079,336; 5,134,119; 5,218,091; 6,072,029; and Wright et ah, Biotechnology 9: 830 (1991); and Archibald et ah Proc. Natl. Acad. Sci. (USA), 87: 5178 (1990), are each herein incorporated by reference for its teaching of AAT sequences, recombinant AAT, and/or recombinant expression of AAT.

As used herein “immunomodulatory drugs or agents”, may refer to agents that act on the immune system, directly or indirectly, e.g., by stimulating or suppressing a cellular activity of a cell in the immune system, e.g., T-cells, B-cells, macrophages, or antigen presenting cells (APC, dendritic cells), or by acting upon components outside the immune system which, in turn, stimulate, suppress, or modulate the immune system, e.g. cytokines, e.g., hormones, receptor agonists or antagonists, and neurotransmitters; immunomodulators can be, e.g., immunosuppressants or immunostimulants.

GvHD

After bone marrow transplantation, T cells present in the graft, either as contaminants or intentionally introduced into the host, attack the tissues of the transplant recipient after perceiving host tissues as antigenically foreign. The T cells produce an excess of cytokines, including TNF-α and interferon-gamma (IFNγ). A wide range of host antigens can initiate GvHD, among them the human leukocyte antigens (HLAs). However, GvHD (also written “GVHD”) can occur even when HLA-identical siblings are the donors. HLA-identical siblings or HLA-identical unrelated donors often have genetically different proteins (called minor histocompatibility antigens) that can be presented by major histocompatibility complex (MHC) molecules to the donor's T-cells, which see these antigens as foreign and so mount an immune response.

GvHD may be classified as acute or chronic GvHD. In the classical sense, acute GvHD is characterized by selective damage to organs and tissues including, but not limited to, the liver, skin (rash), mucosa, and gastrointestinal (GI) tract. Chronic GvHD also attacks the above organs, but over its long-term course is also known to cause damage to the lungs, connective tissue, eyes and exocrine glands. GI GvHD can result in severe intestinal inflammation, sloughing of the mucosal membrane, severe or high-volume diarrhea, gastrointestinal bleeding, abdominal pain, nausea, anorexia, and vomiting. GI GvHD is typically diagnosed via intestinal biopsy.

Acute GvHD is staged as follows: overall grade (skin-liver-gut) with each organ staged individually from a low of 1 to a high of 4. A human subject with grade 4 GvHD usually has a poor prognosis. If the GvHD is severe and requires intense immunosuppression involving steroids and additional agents to get it under control, a subject may develop severe infections as a result of the immunosuppression and may die of infection.

Once it is known whether a patient is at risk of developing severe or fatal GvHD, such knowledge can guide the intensity and duration of treatment and minimize the toxicity associated with chronic steroid administration, as such steroid administration will be of more therapeutic benefit in less severe cases of GvHD. The ability to identify patients who will likely not respond to traditional steroid treatment and who are at particularly high risk for morbidity and mortality related to severe GvHD could guide tailored treatment plans, such as AAT, which may be more effective if introduced early. If the patient is not identified as being at high risk for developing severe GvHD, such a patient is more likely to be suitable for therapeutic regimens designed for less severe GvHD, and may tolerate a more rapid tapering of administered steroids in order to prevent long-term toxicity, infections, and a loss of the graft versus leukemia effect. Follow-up biomarker monitoring in patients at risk of developing severe GvHD could also help decide whether to taper or alter the treatment/prophylaxis, and allow the success of the treatment/prophylaxis to be monitored throughout the duration of therapy.

Regenerating islet-derived 3-alpha (REG3a), a C-type lectin secreted by Paneth cells, was identified as a biomarker specific for lower GI GvHD through an unbiased, in-depth tandem MS-based discovery approach that can quantify proteins at low concentrations. REG proteins act downstream of IL-22 to protect the epithelial barrier function of the intestinal mucosa through the binding of bacterial peptidoglycans. Intestinal stem cells (ISCs) are the principal cellular targets of GvHD in the GI tract, where intestinal flora are critical for amplification of GvHD damage. Without being bound by theory, a leading hypothesis is that ISCs are protected by anti-bacterial proteins, such as REG3α, secreted by neighboring Paneth cells into the crypt microenvironment. If death of ISCs eventually manifests itself as denudation of the mucosa, the patchy nature of GvHD histologic damage may be explained as the lack of mucosal regeneration following the dropout of individual ISCs. REG3a reduces the inflammation of human intestinal crypts in vitro, and its administration protects ISCs and prevents GI epithelial damage in vivo, raising interesting therapeutic possibilities for this molecule.

REG3a protein plasma concentrations correlate with disease activity in inflammatory bowel disease, and can distinguish infectious and autoimmune causes of diarrhea. Without being bound by theory, correlation of mucosal denudation (histologic grade 4) with high REG3a concentrations suggests that microscopic breaches in the mucosal epithelial barrier caused by severe GvHD permit REG3a to traverse into the systemic circulation. The tight proximity of Paneth cells with ISCs, concentrates their secretory contents in that vicinity, so that mucosal barrier disruption caused by stem cell dropout may preferentially allow Paneth cell secretions, including REG3a, to traverse into the bloodstream. It is hypothesized that plasma levels of REG3a may therefore serve as a surrogate marker for the cumulative area of these breaches to GI mucosal barrier integrity, a parameter impossible to measure by individual tissue biopsies. Without being bound by theory, such an estimate of total damage to the mucosal barrier may also help explain the prognostic value of REG3a with respect to therapy responsiveness and NRM.

ST2 is the IL33 receptor, a member of the IL/Toll-like receptor superfamily. ST2 promotes a Th2-type immune response in diseases, such as arthritis and asthma (Kakkar et al., Nature Reviews Drug Discovery 7: 827-40, 2008).

“Acute” as used herein means arising suddenly and manifesting intense severity. With relation to delivery or exposure, “acute” refers to a relatively short duration. “Chronic” as used herein means lasting a long time, sometimes also meaning having a low intensity. With regard to delivery or exposure, “chronic” means for a prolonged period or long-term.

The terms “prevent” or “preventing” includes alleviating, ameliorating, halting, restraining, slowing, delaying, or reversing the progression, or reducing the severity of pathological conditions described above, or forestalling the onset or development of a disease, disorder, or condition for a period of time from minutes to indefinitely. Prevent also means reducing the risk of developing a disease, disorder, or condition.

“Amelioration” or “ameliorate” or “ameliorating” refers to a lessening of at least one indicator, sign, or symptom of an associated disease, disorder, or condition. The severity of indicators may be determined by subjective or objective measures, which are known to those skilled in the art.

“Pre-emptive treatment” refers to administration of AAT before the onset of disease, which refers to the use of AAT in individuals at risk of developing disease and where there may be early signs that emergence of clinically relevant GvHD is imminent. For example, an experimental or predictive serum or cellular biomarker may indicate the optimal time for initiation of pre-emptive GvHD treatment.

In certain embodiments, a subject having GvHD may become resistant to the therapeutic effects of steroids (known as steroid refractory GvHD, or SR GvHD) and alternative agents are required to treat GvHD and the side-effects associated thereto with an alternative agent. In accordance with these embodiments, any steroid used to reduce graft rejection or inhibit GvHD is contemplated herein where a subject can become resistant to its effects. In certain embodiments, glucocorticoids (steroids), for example methylprednisolone or prednisone given at doses of 0.5-2 mg/kg/day, are one frequently used therapy of acute GvHD. Approximately 40% of subjects receiving this agent achieve satisfactory responses, and steroids can be tapered off without significant flares of GvHD. Responses to these agents depend upon the primary organ involvement and the severity of GvHD manifestations in the subject. Severe involvement of the gastrointestinal tract, for example, has proven challenging to treat, with a fatality rate of around 80% in steroid-non-responsive (steroid-refractory, SR) subjects. Various agents, including anti-thymocyte globulin (ATG), monoclonal antibodies, extracorporeal photopheresis (ECP), and other strategies, have been used to treat steroid-refractory GvHD, but have met with only partial success. In certain embodiments disclosed herein, glucocorticoid refractory subjects having advanced-stage GvHD are contemplated for treatment with AAT regimens. In accordance with these embodiments, these subjects can include those refractory to methylprednisolone. In other embodiments, these subjects can be treated with an initial high dose of AAT (or AAT fusion polypeptide) followed by lower doses of AAT.

Any of the embodiments detailed herein may further include one or more therapeutically effective amounts of anti-microbial drugs, anti-inflammatory agents, immunomodulatory agents, or immunosuppressive agents or combination thereof. Non limiting examples of anti-rejection agents/drugs may include for example cyclosporine, azathioprine, corticosteroids, FK506 (tacrolimus), RS61443, mycophenolate mofetil, rapamycin (sirolimus), mizoribine, 15-deoxyspergualin, and/or leflunomide or any combination thereof.

Thus, according to some embodiments, the method of reducing the risk to develop GvHD comprises administering AAT in combination with at least one additional pharmaceutically active agent. According to some embodiments the additional pharmaceutically active agent includes, but is not limited, to any one or more of anti-inflammatory agents, immunomodulatory agents, Janus-associated kinase (JAK) inhibitors, methotrexate, mTOR inhibitors, proteasome inhibitors, anti-inflammatory monoclonal antibodies, cytotoxic monoclonal antibodies, ECP (extracorporeal photopheresis), mesenchymal stem cell-based therapies, cannabis-based therapies and immunosuppressive agents. According to some embodiments, the proteasome inhibitors comprise bortezomib. According to some embodiments, the anti-inflammatory monoclonal antibodies comprise any one or more of natalizumab, vedolizumab and basiliximab. According to some embodiments, the cytotoxic monoclonal antibodies comprise brentuximab. According to some embodiments, the immunosuppressive agents comprise steroids. According to some embodiments, the immunosuppressive agents are nonsteroidal immunosuppressive agents. Non limiting examples of nonsteroidal immunosuppressive agents may include cyclosporine, azathioprine, FK506 (tacrolimus), RS-61443, mycophenolate mofetil, rapamycin (sirolimus), mizoribine, 15-deoxyspergualin, and leflunomide. According to some embodiments, the JAK inhibitors comprise any one or more of ruxolitinib and itacitinib. According to some embodiments, the mTOR inhibitors comprise sirolimus.

The term “dosage” as used herein refers to the amount, frequency and duration of AAT which is given to a subject during a therapeutic period.

The term “dose” as used herein, refers to an amount of AAT which is given to a subject in a single administration.

The terms “multiple-variable dosage” and “multiple dosage” are used herein interchangeably and include different doses of AAT administration to a subject and/or variable frequency of administration of the AAT for therapeutic treatment. The terms “multiple dose regimen” or “multiple-variable dose regimen” describe a therapy schedule which is based on administering different, or similar, amounts (doses) of AAT at various time points throughout the course of therapy.

“Inhalation” refers to a method of administration of a compound that delivers an effective amount of the compound so administered or delivered to the tissues of the lungs or lower respiratory tract by inhalation of the compound by the subject, thereby drawing the compound into the lung. As used herein, “administration” is synonymous with “delivery”.

The term “eFlow nebulizer” refers to the nebulizer disclosed in international application WO 01/34232. The term “inhalation nebulizer” refers to a nebulizer comprising the basic elements of the eFlow nebulizer and any equivalent nebulizer. The terms “pulmonary delivery” and “respiratory delivery” refer to delivery of AAT to a patient by inhalation through the mouth or nose and into the lungs.

The term “dry powder” refers to a powder composition that contains finely dispersed dry particles that are capable of being dispersed in an inhalation device and subsequently inhaled by a subject.

As used herein, the term “biomarker” refers to an indicator of, for example, a pathological state of a subject, which can be detected in a biological sample taken from the subject. Biomarkers include DNA-based, RNA-based and protein-based molecular markers.

As used herein, the term “diagnosis” refers to the identification or classification of a molecular or pathological state, disease or condition. For example, “diagnosis” can refer to identification of a particular type of a condition (such as graft-versus host disease (“GvHD”)).

As used herein, the term “aiding diagnosis” refers to methods that assist in making a clinical determination regarding the presence, or nature, of a particular type of symptom or condition of a condition (such as GvHD). For example, a method of aiding diagnosis of a condition (such as GvHD) can include measuring the expression of certain genes in a biological sample taken from an individual.

As used herein, the term “prognosis” is used herein to refer to the categorization of patients by degree of risk for a disease (such as GvHD) or progression of such disease. A “prognostic marker” refers to an assay that categorizes patients by degree of risk for disease occurrence or progression.

As used herein, the term “sample” refers to a composition that is obtained or derived from a subject of interest and that contains a cellular and/or other molecular entity that is to be characterized and/or identified, for example based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. A “tissue” or “cell sample” refers to a collection of similar cells obtained from a tissue of a subject or patient. The source of the tissue or cell sample may be blood or any blood constituents (e.g., whole blood, plasma, serum) from the subject. The tissue sample can also be primary or cultured cells or cell lines. Optionally, the tissue or cell sample is obtained from a disease tissue/organ. The tissue sample can contain compounds which are not naturally intermixed with the tissue in nature such as preservatives, anticoagulants, buffers, fixatives, nutrients, antibiotics, and the like.

The term “subject” is used interchangeably herein with “patient” to refer to an individual to be treated. The subject is a mammal (e.g., human, non-human primate, rat, mouse, cow, horse, pig, sheep, goat, dog, cat, etc.). The subject can be a clinical patient, a clinical trial volunteer, an experimental animal, etc. The subject can be suspected of having or being at risk of having a condition (such as GvHD) or be diagnosed with a condition (such as GvHD). According to one embodiment, the subject to be treated according to this invention is a human.

Pharmaceutical Compositions

According to certain embodiments, AAT is administered in the form of a pharmaceutical composition. As used herein, the term “pharmaceutical composition” refers to a preparation of AAT with other chemical components such as pharmaceutically acceptable carriers and excipients. The purpose of a pharmaceutical composition is to facilitate administration of an active ingredient to an organism, and enhance its stability and turnover.

Any available AAT as is known in the art, including plasma-derived AAT and recombinant AAT can be used according to the teachings of the present invention. According to certain exemplary embodiments, the AAT is produced by the method described in U.S. Pat. No. 7,879,800, granted to the Applicant of the present invention.

The term “pharmaceutically acceptable” means approved by a regulatory agency of the Federal or a state government or listed in the U. S. Pharmacopeia or other generally recognized pharmacopeia for use in animals, and more particularly in humans.

The term “carrier” refers to a diluent or vehicle that does not cause significant irritation to an organism and does not abrogate the biological activity and properties of the administered compound. An adjuvant is included under these phrases. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when the pharmaceutical composition is administered intravenously. Saline solutions, isotonic buffers and physiological pH and aqueous dextrose and glycerol solutions can also be employed as liquid carriers, particularly for injectable solutions.

The pharmaceutical compositions of the invention can further comprise an excipient. Herein, the term “excipient” refers to an inert substance added to a pharmaceutical composition to further facilitate administration of an active ingredient. Suitable pharmaceutical excipients include starch, glucose, lactose, sucrose, trehalose, gelatin, malt, rice, flour, chalk, silica gel, sodium stearate, glycerol monostearate, talc, sodium chloride, dried skim milk, glycerol, propylene glycol, water, lipids, phospholipids, ethanol, and the like. The composition, if desired, can also contain minor amounts of wetting or emulsifying agents, or pH buffering agents such as acetates, citrates, or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned.

The pharmaceutical compositions of the present invention can be manufactured by processes well known in the art, e.g., by means of conventional mixing, dissolving, granulating, dragee-making, levigating, emulsifying, encapsulating, entrapping, spray drying, or lyophilizing processes.

According to certain exemplary embodiments, pharmaceutical compositions, which contain AAT as an active ingredient, are prepared as injectable, either as liquid solutions or suspensions, however, solid forms, which can be suspended or solubilized prior to injection, can also be prepared. According to additional exemplary embodiments the AAT-containing pharmaceutical composition is formulated in a form suitable for inhalation. According to yet additional embodiments, the AAT-containing pharmaceutical composition is formulated in a form suitable for subcutaneous administration. From the patient point of view multiple injections are not a favorable treatment, and thus it may be replaced by slow and/or controlled release subcutaneous administration. Any other forms of slow and/or controlled release are also explicitly encompassed within the scope of the present invention.

The compositions can also take the form of emulsions, tablets, capsules, gels, syrups, slurries, powders, creams, depots, sustained-release formulations and the like.

Methods of introduction of a pharmaceutical composition comprising AAT include, but are not limited to, intravenous, subcutaneous, intramuscular, intraperitoneal, oral, topical, intradermal, transdermal, intranasal, epidural, ophthalmic, vaginal, and rectal routes. The pharmaceutical compositions can be administered by any convenient route, for example by infusion or bolus injection, by absorption through epithelial linings (e.g., oral mucosa, rectal, and intestinal mucosa, etc.), and may be administered together with other therapeutically active agents. The administration may be localized, or may be systemic. Pulmonary administration can also be employed, e.g., by use of any type of inhaler or nebulizer.

Pharmaceutical compositions for use in accordance with the present invention may be formulated in a conventional manner using one or more physiologically acceptable carriers comprising excipients and auxiliaries, which facilitate processing of the active ingredients into preparations that can be used pharmaceutically. Proper formulation is dependent upon the route of administration chosen.

For injection, the active ingredients of the pharmaceutical composition may be formulated in aqueous solutions, typically in physiologically compatible buffers such as Hank's solution, Ringer's solution, or physiological salt buffer. For transmucosal or transdermal administration, penetrants appropriate to the barrier to be permeated are used in the formulation. Such penetrants are generally known in the art.

For oral administration, the pharmaceutical composition can be formulated readily by combining the active ingredients with pharmaceutically acceptable carriers well known in the art. Such carriers enable the pharmaceutical composition to be formulated as tablets, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin, as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane, or carbon dioxide. In the case of a pressurized aerosol, the dosage may be determined by providing a valve to deliver a metered amount. Capsules and cartridges of, for example, gelatin for use in a dispenser may be formulated containing a powder mix of the compound and a suitable powder base, such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with, optionally, an added preservative. The compositions may be suspensions, solutions, or emulsions in oily or aqueous vehicles, and may contain formulatory agents such as suspending, stabilizing, and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

Alternatively, the active ingredient may be in powder form for constitution with a suitable vehicle, e.g., a sterile, pyrogen-free, water-based solution, before use.

The pharmaceutical composition of the present invention may also be formulated in rectal compositions such as suppositories or retention enemas, using, for example, traditional binders and carriers such as triglycerides, microcrystalline cellulose, gum tragacanth or gelatin.

According to certain exemplary embodiments, the AAT-containing pharmaceutical composition used according to the teachings of the present invention is a ready-to-use solution. According to further exemplary embodiments, the AAT-containing pharmaceutical composition is marketed under the trade name Glassia®.

Therapeutic Methods

In one embodiment, methods provide for pre-emption treatment of GvHD with an AAT-containing composition are disclosed herein. In accordance with these embodiments, compositions disclosed herein can include a compound capable of inhibiting at least one serine protease or having other activity, for example, AAT or carboxyterminal peptide or fusion polypeptide thereof, or recombinant thereof, or analog thereof. In another embodiment, methods and compositions described herein can be useful in the therapeutic treatment of graft rejection associated side effects. In a yet another embodiment, late-stage GvHD associated side effects can be reduced or prevented by administration of the compositions as disclosed herein, in order to ameliorate, reduce, or eliminate one or more symptoms, side-effects, or one or more signs, or prior to the onset of one or more severe symptoms, or even mortality due to grade 3 or 4 gut involvement. It is contemplated herein that the present compositions and methods can be used to treat a subject requiring chronic therapy.

The term ‘treatment of GvHD’ as used herein includes, but is not limited to, prevention of GvHD, preventing SR GvHD, preventing acute GvHD, preventing chronic GvHD, reducing GvHD associated symptoms, preventing GvHD symptoms, improving survival rates, reducing cumulative incidence of severe GI GvHD stage 3 or 4, reducing cumulative incidence of chronic GvHD, reducing cumulative incidence of steroid refractory GvHD, reducing cumulative incidence of non-relapse mortality (NRM), preventing NRM, reducing the cumulative incidence of relapse, preventing relapse, reducing organ damage associated with GvHD, preventing organ damage, attenuating the staging (severity) of organ damage and increasing overall survival. According to some embodiments, treatment of GvHD refers to treatment of acute GvHD. According to some embodiments, treatment of GvHD refers to treatment of chronic GvHD.

The following examples are included to demonstrate preferred embodiments of the invention. It should be appreciated by those of skill in the art that the techniques disclosed in the following examples represent techniques discovered by the inventors to function well in the practice of the invention, and thus can be considered to constitute preferred modes for its practice. However, those of skill in the art should, in light of the present disclosure, appreciate that many changes can be made in the specific embodiments, which are disclosed and still obtain a like or similar result without departing from the spirit and scope of the invention.

### EXAMPLES

### Example 1: A Proof of Concept Pilot Trial of Alpha-1-Antitrypsin for Pre-Emption of Steroid-Refractory Acute GvHD—Preliminary Results

1. To assess the feasibility, safety and efficacy of alpha-1-antitrypsin (AAT) as pre-emptive therapy in patients at high risk for the development of steroid-refractory GvHD.

2. To generate an estimate of the 100 day incidence of clinically relevant GvHD states including steroid-refractory GvHD, grade II-IV GvHD, and grade III-IV in patients at high risk for the development of steroid-refractory GvHD treated with AAT.

3. To generate a preliminary estimate of the rates of non-relapse mortality, relapse, and survival in patients at high risk for the development of steroid refractory GvHD treated with AAT.

Number of subjects: 30 subjects. The characteristics of the subjects and matched controls are shown in Table 1. The control cohort (matched historical control taken from the MAGIC patient pool) was well matched to the study subjects with the exception that fewer controls received tacrolimus and sirolimus as GVHD prophylaxis compared to study subjects.

TABLE 1
Patient characteristics

| | | | | |
|---|---|---|---|---|
| Parameter | AAT | (n = 30) | Control | (n = 90) |

| | | |
|---|---|---|
| Years | 2018-2019 | 2015-2019 |

Day high risk

| | | | | |
|---|---|---|---|---|
| Day 7 | 19 | (63%) | 57 | (63%) |
| Day 14 | 11 | (37%) | 33 | (37%) |
| Median Age | 51 | (20-76) | 60 | (19-76) |

GVHD Prophylaxis, n (%)

| | | | | |
|---|---|---|---|---|
| CNI/MTX | 14 | (47%) | 42 | (47%) |
| CNI/MMF | 0 | (0%) | 20 | (22%) |
| CNI/sirolimus | 15 | (50%) | 23 | (26%) |

| | | | |
|---|---|---|---|
| Cyclophosphamide | 0 | 4 | (4%) |
| based | | | |

| | | | | |
|---|---|---|---|---|
| Other | 1 | (3%) | 1 | (1%) |

Donor Type, n (%)

| | | | | |
|---|---|---|---|---|
| Related | 3 | (10%) | 9 | (10%) |
| Unrelated | 27 | (90%) | 81 | (90%) |

HLA Match, n (%)

| | | | | |
|---|---|---|---|---|
| Match | 29 | (97%) | 87 | (97%) |
| Mismatch | 1 | (3%) | 3 | (3%) |

Graft Source, n (%)

| | | | | |
|---|---|---|---|---|
| Bone marrow (BM) | 5 | (17%) | 11 | (12%) |
| Peripheral blood | 25 | (83%) | 79 | (88%) |
| stem cells (PBSC) | | | | |

Condition Regimen Intensity, n (%)

| | | | | |
|---|---|---|---|---|
| Full | 11 | (37%) | 40 | (44%) |
| Reduced | 19 | (63%) | 50 | (56%) |

ATG, n (%)

| | | | | |
|---|---|---|---|---|
| Yes | 6 | (20%) | 31 | (34%) |
| No | 24 | (80%) | 59 | (66%) |

Indication for HCT, n (%)

| | | | | |
|---|---|---|---|---|
| Acute Leukemia | 14 | (47%) | 39 | (43%) |
| Lymphoma | 1 | (3%) | 13 | (15%) |
| MDS/MPN | 13 | (43%) | 28 | (31%) |

| | | | |
|---|---|---|---|
| Other Malignant | 0 | 6 | (7%) |

| | | | | |
|---|---|---|---|---|
| Other Nonmalignant | 2 | (7%) | 4 | (4%) |
| Median Day of | 30.5 | (12-73) | 32 | (10-162) |
| GVHD Diagnosis | | | | |

Day high risk—the day the marker evaluation indicated that the patient is at high risk; CNI, calcineurin inhibitor; MTX, methotrexate; MMF, mycophenolate; TG; anti-thymocyte globulin; HCT, hematopoietic cell transplantation; MDS/MPN, myelodysplastic syndromes/myeloproliferative neoplasms; donor is family related to acceptor/transplanted;

Inclusion Criteria:

1. High risk prediction score as determined by the MAGIC algorithm at either day 7 or day 14 post HCT.

2. Any donor type (e.g., related, unrelated) or stem cell source (bone marrow, peripheral blood, cord blood).

3. Donor and recipient match each other for at least 7/8 HLA-loci (HLA-A, B, C, and DR)

4. Subjects receiving either non-myeloablative or myeloablative or reduced intensity conditioning regimen are eligible.

5. GvHD prophylaxis must include a calcineurin inhibitor combined with methotrexate or mycophenolate.

6. The use of serotherapy to prevent GvHD (e.g., antithymocyte globulin) prior to day 3 post-HCT is permitted.

7. Age 18-75 years

8. Direct bilirubin must be <2 mg/dL within 3 days of enrollment unless the elevation is known to be due to Gilbert syndrome.

9. ALT/SGPT and AST/SGOT must be <5× the upper limit of the normal range within 3 days of enrollment.

Exclusion Criteria:

1. Patients who develop acute GvHD prior to start of study drug.

2. Patients at very high risk for primary disease relapse post HCT defined as a very high disease risk index.

3. Patients participating in a clinical trial where prevention of GvHD is the primary endpoint.

4. Uncontrolled active infection (i.e., progressive symptoms related to infection despite treatment or persistently positive microbiological cultures despite treatment or any other evidence of severe sepsis).

5. Patients who are pregnant.

6. Patients on dialysis within 7 days of enrollment.

7. Patients requiring mechanical respiratory support or patients requiring oxygen supplementation exceeding 40% Fi02 within 14 days of enrollment.

8. Patients receiving an investigational agent within 30 days of enrollment However, the Principal Investigator (PI) may approve prior use of an investigational agent if the agent is not expected to interfere with the safety or the efficacy of alpha-1-antitrypsin

9. History of allergic reaction to alpha-1-antitrypsin.

Dose, Route, Regimen: Alpha-1-antitrypsin (Glassia®) 90 mg/kg intravenous on day 0, then 45 mg/kg twice weekly for 15 more doses (total of 16 doses over 8 weeks).

Glassia® supplied as single use vials, each containing approximately 1 gram of functional Alpha-1-PI (AAT) in 50 mL of a carrier solution comprising phosphate-buffered saline solution and a sterile filter needle.

Duration of AAT Treatment: Eight Weeks

Statistical Methodology: This was a proof of concept pilot study intended to determine whether there is sufficient evidence to warrant further study of a pre-emptive treatment with AAT. The expected incidence of steroid refractory (SR) GvHD by day 100 in patients who are high risk at either day 7 or 14, is 28%. We assumed that the incidence of steroid refractory GvHD by day 100 among high risk patients treated pre-emptively with AAT will be reduced from 28% to 15% as a clinically meaningful incidence. Based on this assumption, a sample size of 30 patients was calculated to achieve 85% statistical power to detect the aforementioned 13% improvement (reduction in the incidence of steroid refractor (SR) GvHD by day 100 from 28% to 15%) using a one-sided exact test with a target significance level of 0.23. Observing 6 or fewer cases of SR GvHD, considered this approach to be sufficiently promising to warrant further study.

Primary Endpoint

The end point was set to observe 6 or fewer cases of steroid refractory GvHD by day 100 post HCT (counting from the transplantation date). GvHD is defined as steroid refractory if a complete response (CR) or partial response (PR) is not achieved by day 28 of (i) systemic steroid treatment; or (ii) if additional immunosuppression beyond steroids was given for treatment of GvHD, prior to day 28 from initiation of steroid treatment upon diagnosis of GvHD. As shown below, the primary study end point was met.

Secondary Endpoints:

1. Overall survival at 6 months

2. Cumulative incidence of NRM at 6 months and 1 year

3. Relapse rate

4. 100 days incidence of clinically relevant GvHD states including steroid-refractory GvHD, grade II-IV GvHD, and grade II-IV GvHD

5. For patients who develop GvHD prior to day 100 post-HCT, the overall response rate (CR+PR) 28 days after initiation of systemic steroid treatment. PR is defined as improvement in one or more organs involved with GvHD symptoms without progression in others. For a response to be scored as PR on day 28, the patient must be in PR on day 28 and have had no intervening systemic therapy for acute GvHD other than steroids

6. Cumulative incidence of severe GI GvHD stage 3 or 4 by day 100 post-HCT

7. Cumulative incidence of chronic GvHD requiring systemic steroid treatment by one year

8. Number of serious infections (defined as grade 3 by the Blood and Marrow Transplant Clinical Trials Network)

Subject Screening and Registration Procedures

To be eligible for this study, patients had to have a high MAGIC GvHD risk score (also termed ‘high MAP score’, ‘high risk’ or ‘HR’) on day 7 or 14 post-HCT. Patients were recruited from centers participating in the Mount Sinai Acute GvHD International Consortium (MAGIC) where the procedures for obtaining screening samples for biomarker scoring are already established. Consented patients were registered into the remote data entry system using a unique study number assigned by the MAGIC DCC. Serum (5 mL) was collected from patients on day 7 post-HCT (+/−1 day) and shipped to the Mount Sinai GvHD laboratory for early AM arrival. Once received in the laboratory, the GvHD biomarkers used to assign the MAGIC GvHD risk score were measured by ELISA using standard technical procedures.

Patients who were found to be at low risk on day 7, who have not already developed GvHD requiring systemic treatment, and who met the other eligibility criteria were re-screened on day 14 (+/−1 day). Patients who were found to be at low risk on day 7 but at high risk on day 14 were also eligible for this study provided they have not already developed GvHD requiring systemic treatment. Only patients with confirmed high MAGIC GvHD risk scores were eligible to enroll in the clinical trial. Patient registration for this trial was centrally managed by the MAGIC Data Coordinating Center of the Icahn School of Medicine at Mount Sinai.

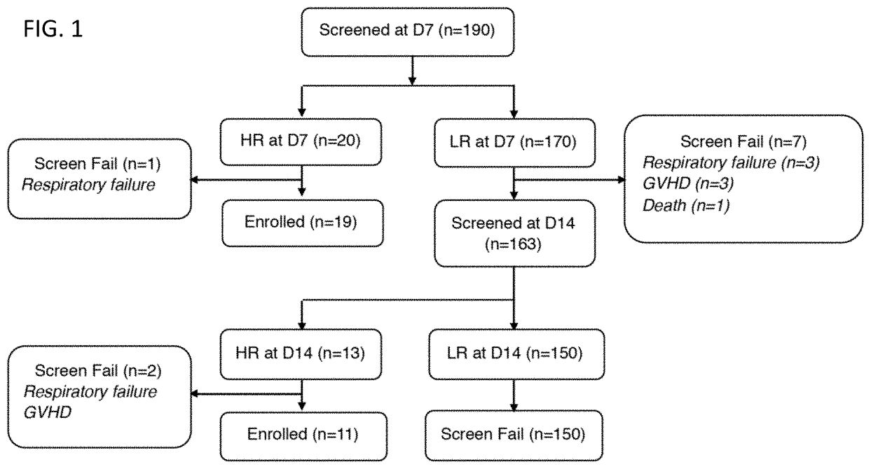

A consort diagram depicting the screening process is shown in FIG. 1. One-hundred ninety patients were screened at day 7 and twenty were found to be at high risk by MAP. One patient with a high MAP on day 7 developed respiratory failure the next day and was excluded. Of the 170 patients who were found to be at low risk at day 7 and monitored for another week, seven were excluded because they developed either GVHD (n=3), respiratory failure (n=3), or died (n=1). Of the remaining 163 patients who were re-screened on day 14, thirteen additional patients were found to be at high risk by MAP. Two of these patients were excluded because they developed either GVHD (n=1) or respiratory failure (n=1) by the time MAP results were reported the following day. Altogether, 33/190 patients (17%) had high MAPs early post-HCT and 30 patients met all eligibility criteria and were enrolled in the clinical trial.

Duration of Follow-Up

Patients were followed until 1 year post-HCT, withdrawal of consent, or until death, whichever occurred first. HCT patients were followed closely and frequent clinical evaluations were the norm. The following outlines the minimum frequency of follow-up evaluations.

During the first 8 weeks of participation (i.e., through the last dose of alpha-1-antitrypsin), patients were seen twice weekly for study drug administration. GvHD staging was performed approximately weekly through day 100 post-HCT as part of the standard of care for HCT recipients. Patients were evaluated at least monthly until 6 months post-HCT and then again at one year post-HCT. If GvHD developed during the first six months post-HCT, staging and treatment response were evaluated at least weekly for four weeks as part of the standard of care for patients who developed GvHD.

GvHD Clinical Staging

GvHD clinical staging was done according to the established criteria used for Blood and Marrow Transplant Clinical Trials Network GvHD staging (modified Glucksberg criteria).

Overall Clinical Grade:

Grade 0 No Stage 1-4 of any organ

Grade I Stage 1-2 skin and no liver or GI involvement

Grade II Stage 3 skin and/or Stage 1 liver and/or Stage 1 GI

Grade III Stage 0-3 skin with Stage 2-3 liver and/or Stage 2-3 GI

Grade IV Stage 4 in any target organ (skin, liver, GI)

Endpoint and Response Criteria

### Definitions

Evaluable for response: Safety, tolerability, and efficacy of AAT were assessed from the initiation of the first AAT treatment

Complete Response (CR): All evaluable organs (skin, liver, GI tract) stage 0. For a response to be scored as CR on day 28, the patient must have been in CR on that day and have had no intervening additional GvHD therapy.

Partial Response (PR): An improvement in one or more organs involved with GvHD symptoms without worsening in others. For a response to be scored as PR on day 28, the patient must have been in PR on that day and have had no intervening additional GvHD therapy.

No response (NR) to steroid treatment: All responses that were not CR or PR. Patients who received any systemic GvHD therapy other than the continuation or modification of GvHD prophylaxis (Table 1), systemic steroids, and topical/non-absorbable oral steroid therapy, were scored as NR on day 28 regardless of organ staging.

Proportion of CR and CR+PR

CR and PR on day 28 after systemic steroid treatment began were scored in comparison to the patient's acute GvHD staging on the day systemic steroid treatment began.

Steroid refractory GvHD

Patients who were scored as no response on day 28 of systemic steroid treatment for GvHD or who received additional systemic immunosuppression prior to day 28 (i.e., no response), were considered steroid refractory. Escalations of steroid doses during treatment for acute GvHD were not considered valid for the definition of steroid refractory GvHD.

Steroid Discontinuation

The date of discontinuation of steroid therapy was recorded.

Lines of GvHD Therapy

Systemic steroids were the first line of GvHD treatment. Any systemic immunosuppression treatment given in addition to steroid therapy for acute GvHD was considered 2nd line therapy and considered a failure to respond to steroid treatment. Resumption or changes in GvHD prophylaxis (e.g., substitution of mycophenolate for tacrolimus due to posterior reversible encephalopathy syndrome (PRES)) were not considered new lines of therapy. Topical steroids and non-absorbable oral steroids were not considered new lines of therapy.

Non-Relapse Mortality (NRM)

Any death that occurred after HCT and was not attributable to relapse of the underlying disease was considered a non-relapse death.

Chronic GvHD

The occurrence of chronic GvHD as defined by NIH consensus criteria requiring systemic treatment, including date of diagnosis, was recorded.

Relapse

Relapse, including date of relapse, of the underlying malignancy was recorded.

Data Analyses Plans

The primary endpoint was incidence of steroid-refractory GvHD by day 100. Death, lack of GvHD response to systemic steroids by day 28 of treatment, or initiation of additional systemic immunosuppressive therapy for GvHD were considered failures for this endpoint. The incidence of steroid-refractory GvHD by day 100 in the study patients was compared to the historical control rate of 28%, as detailed above.

Secondary outcomes such as the overall response rate (CR+PR), the incidence of severe GI GvHD (stage 3 or 4), non-relapse mortality, relapse rates, and overall survival, were estimated and compared to the historical controls.

Continuous variables were summarized using standard summary statistics such as number of observations (n), mean, standard deviation (SD), minimum and maximum values, median, and 1st and 3rd quartiles. Categorical variables were summarized in frequency tables as counts and percentages (not shown).

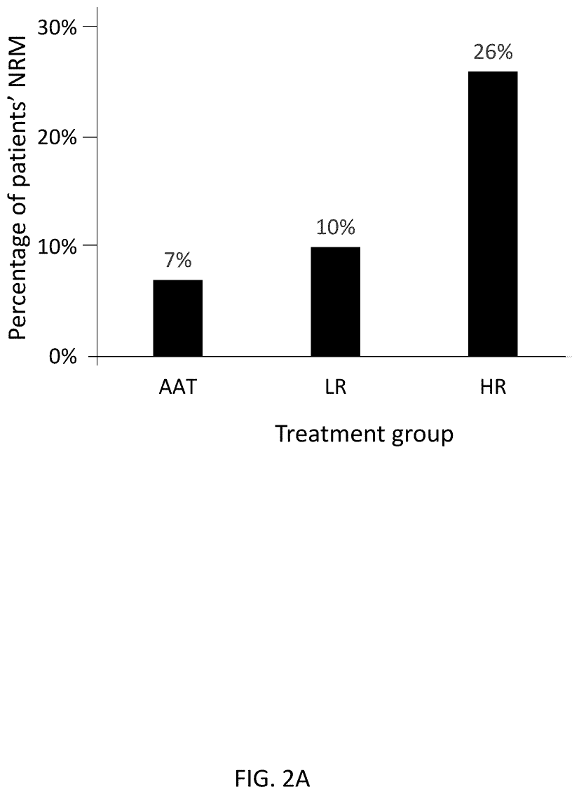

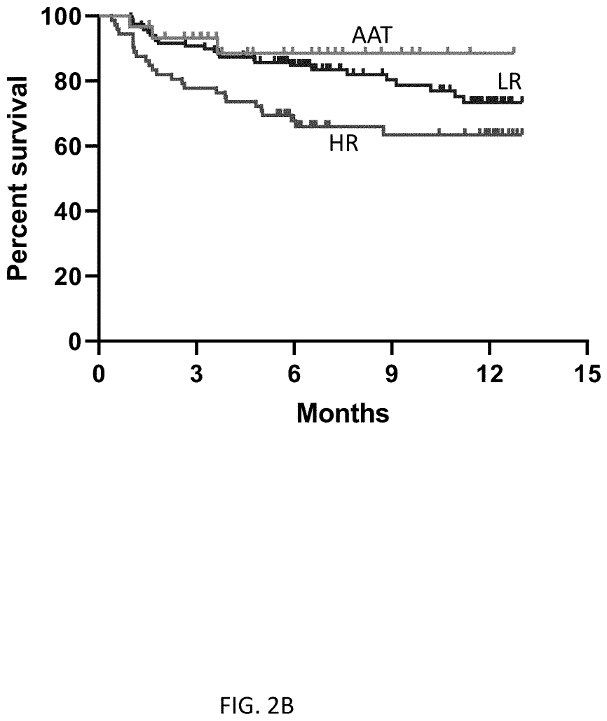

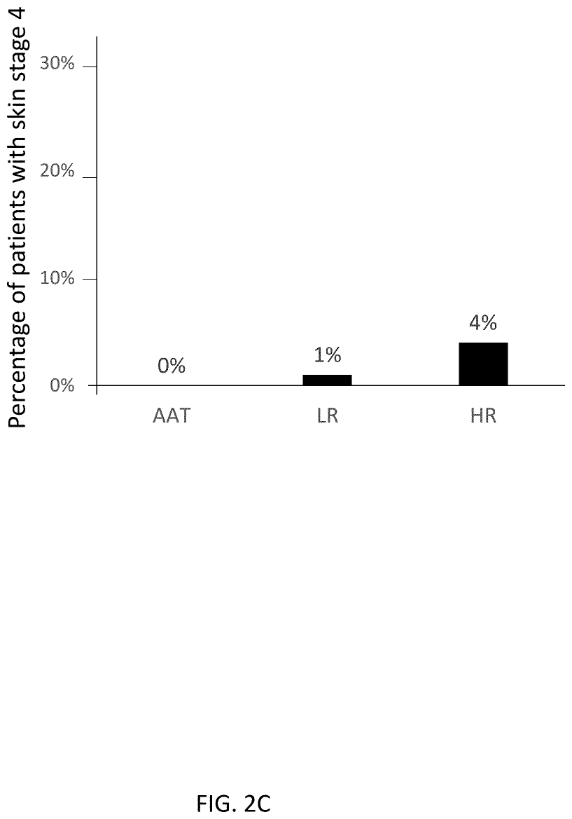

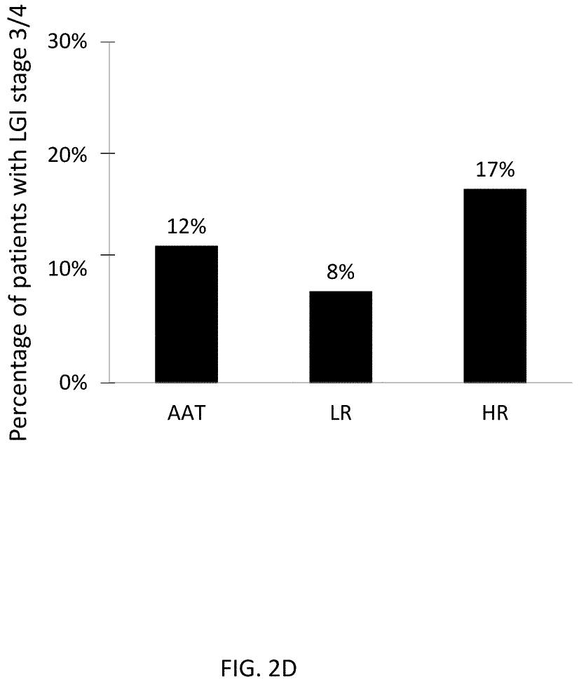

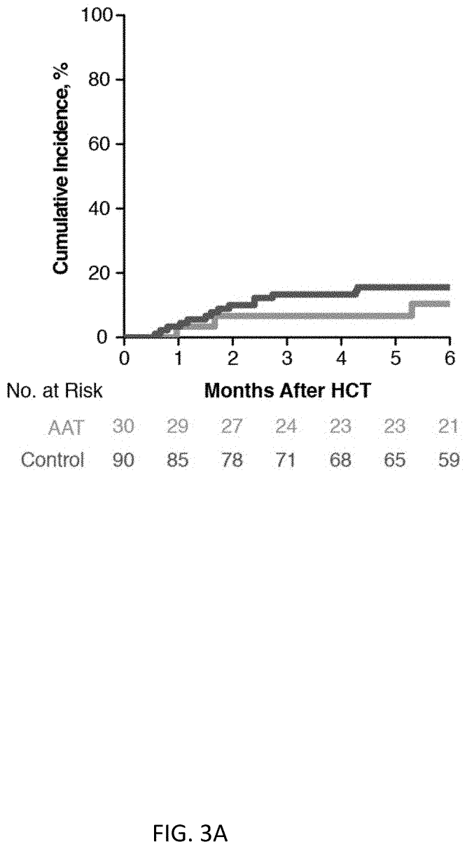

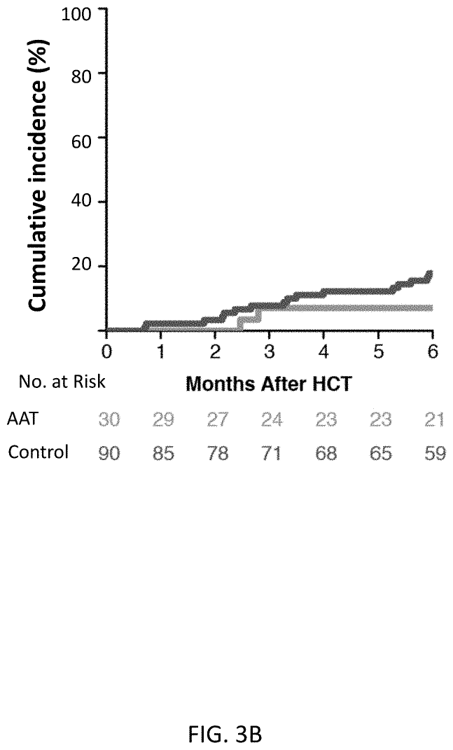

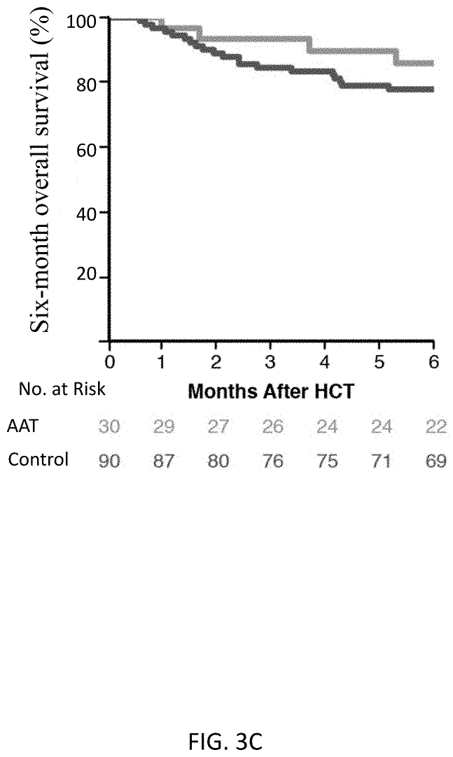

The cumulative incidence of non-relapse mortality was estimated by Gray's method and disease relapse was considered as a competing risk (results are shown in FIGS. 3A and 3B, respectively). Disease free (not shown) and overall survival (FIGS. 2B and 3C), defined as the time from the transplantation to death or to last follow-up if alive, was estimated by the method of Kaplan-Meier and the probability curves and 95% confidence intervals were provided based on the method of Brookmeyer and Crowley.

Preliminary Clinical Outcome

Preliminary results of seven (7) patients are presented in Table 2. These results were accumulated upon identifying a first patient who developed SR GvHD. The results indicate that out of the seven recorded patients thus far, four were diagnosed as suffering from GvHD (patient no. 1, 2, 3 and 5). Two of these patients received steroid treatment at the day of GvHD diagnosis based on their organ staging (skin staging 3, patient no. 1 and 5). Only one patient (patient no. 1) developed SR GvHD (and hence received additional 2^{nd} treatment, not shown). However, all other patients diagnosed with GvHD did not develop SR GvHD following treatment with AAT. These preliminary results were encouraging and hence the clinical study continued as planned, accruing all 30 patients.

TABLE 2
AAT Study Data Summary (as of 6 Dec. 2018)

| | | | No of | | | | | Steroid |
|---|---|---|---|---|---|---|---|---|
| Patient | | Biomarkers | AAT | | | Max | Organ | refactory |
| ID | BMT date | HS 7/14 | Doses | GvHD DX date | GvHD TX date | Grade | staging | GvHD |
| 1 | 10 Aug. 2018 | 7 | 16 | 22 Oct. 2018 | 22 Oct. 2018 | 2 | 3, 0, 0, 0 | Yes |
| 2 | 20 Sep. 2018 | 14 | 15 | 16 Oct. 2018 | No | 2 | 0, 0, 1, 0 | |
| 3 | 11 Oct. 2018 | 14 | 8 | 16 Nov. 2018 | No | 2 | 0, 0, 1, 0 | |
| 4 | 6 Nov. 2018 | 7 | 4 | No | No | 0 | 0, 0, 0, 0 | |
| 5 | 26 Oct. 2018 | 7 | 7 | 27 Nov. 2018 | 27 Nov. 2018 | 2 | 3, 0, 0, 0 | |
| 6 | 13 Nov. 2018 | 14 | 2 | No | No | 0 | 0, 0, 0, 0 | |
| 7 | 23 Nov. 2018 | 7 | 1 | No | No | 0 | 0, 0, 0, 0 | |

BMT—bone marrow transplant; HS—high MAP scoring; DX—diagnosis.

### Example 2. Intermediate Clinical Results

Interim data was randomly collected during the study and the percentage of control (not treated with AAT) patients with high risk (HR) and low risk (LR) to develop SR GVHD was evaluated and compared to the percentage of high risk patients treated with AAT.

The interim results indicate that preemptive treatment with AAT reduced NRM from the expected 26% to 7%, namely, less than 10% which is a lower rate than the NRM observed in low risk patients (FIG. 2A).

The overall survival also demonstrated improved survival rates in HR patients receiving pre-emptive AAT treatment compared to control HR patients not treated with AAT (FIG. 2B). Thus, AAT administration changes the course of the disease from the high mortality pattern as observed in control high risk patients towards an attenuated mortality rate (FIGS. 2A and 2B).

In addition, high risk control patients that were not treated with AAT developed GvHD with severe skin involvement (stage 4) which was not observed in patients treated with AAT (FIG. 2C). Furthermore, 17% of the high-risk control patients developed severe lower gastrointestinal (LGI) involvement (stage 3/4) while only 12% of the high-risk patients treated with AAT developed stage 3/4 LGI (FIG. 2D). Thus, the interim data exhibited the beneficial effect exerted by AAT treatment on GVHD symptoms in high risk patients, and particularly, demonstrated improved survival in AAT treated high risk patients.

### Example 3. Clinical Study Results

Significant (grade 3 or higher) treatment-emergent adverse events (AE) are shown in Table 3. The most common adverse events observed during AAT therapy were pulmonary complications: acute respiratory distress syndrome (ARDS; n=2), idiopathic pneumonia syndrome (IPS; n=2), diffuse alveolar hemorrhage (DAH; n=1), and pulmonary edema (n=1). AAT therapy was considered related to DAH in one subject who experienced the complication twice within hours of consecutive infusions. However, the incidence of respiratory failure was similar in study subjects and controls (13% vs 19%, p=0.59). Four subjects (13%) experienced reactions known to occur with AAT infusions, including grade 1 pruritus, grade 2 supraventricular tachycardia, grade 3 urticaria and grade 3 facial edema. One subject discontinued AAT infusions due to recurrent episodes of urticaria and facial edema despite pre-medication with diphenhydramine and acetaminophen; the other three subjects experienced no additional reactions with subsequent infusions.

TABLE 3
Cause of 6-month non-relapse mortality

| | CTCAE | CTCAE | CTCAT |
|---|---|---|---|
| AE type | Grade 3 | Grade 4 | Grade 5 |
| Cardiac disorder | 1 | 0 | 0 |
| Engraftment syndrome | 2 | 0 | 0 |
| Gastrointestinal disorder | 1 | 0 | 0 |
| Headache | 1 | 0 | 0 |
| Hypertension | 1 | 0 | 0 |
| Bacterial infection | 2 | 1 | 0 |
| Fungal infection | 0 | 1 | 1 |
| Infusion reaction | 2 | 0 | 0 |
| Pulmonary event | 2 | 2 | 2 |
| Thrombotic microangiopathy | 1 | 0 | 0 |
| Weakness/Fatigue | 1 | 0 | 0 |

CTCAE—Common Terminology Criteria for Adverse Events

Therapy with AAT consisted of twice weekly infusions for 8 weeks with planned early cessation in patients requiring additional treatment for SR GVHD. AAT Treatment was begun at a median of 1 day (range 0-3 days) from confirmation of the high risk MAP score. Twenty-four patients (80%) received all protocol defined doses of AAT. Six subjects discontinued therapy early due to infusion reaction (n=1), adverse events (ARDS, n=2, DAH, n=1), and physician discretion (n=2).

Advantageously, treatment with AAT induced a decrease in the six-month cumulative incidence of non-relapse mortality in study subjects compared to controls (10% vs. 16%, respectively; FIG. 3A). All causes of NRM are summarized in Table 4. Furthermore, treatment with AAT reduced the six-month cumulative incidence of the primary disease relapse compared to controls (7% vs. 18%, respectively; FIG. 3B) and, as a result, 6-month overall survival was higher with AAT treated patients compared to controls (86% vs. 78%, respectively; FIG. 3C). Thus, AAT induced beneficial effects post transplantation, by reducing, and even preventing, non-relapse mortality and, hence increasing overall survival.

TABLE 4
Cause of six-mouth non-relapse mortality

| Cause | AAT (n = 30) | Control (n = 90) |
|---|---|---|
| aGVHD | 1 (3.3%) | 6 (6.7%) |
| ^{A}Infection | 1 (3.3%) | 3 (3.3%) |
| ^{B}Other | 1 (3.3%) | 5 (5.6%) |

^{A}Infection-AAT: pneumocystis jirovecii pneumonia/aspergilus pneumonia (1). Control: disseminated candidemia (2), septic shock (1).
^{B}Other cause of death-AAT: Idiopathic pneumonia syndrome (1). Control: Myocardial infarction (1), intracranial hemorrhage (1), veno-occlusive disease (1), multi-organ failure (1), unknown (1).

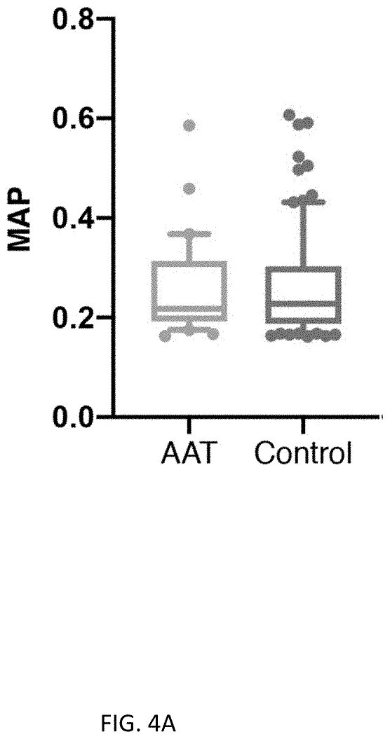

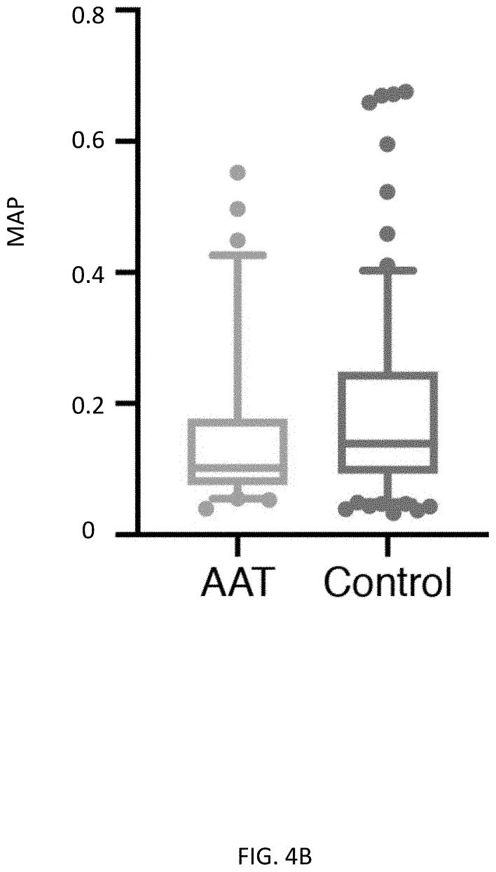

The beneficial effect of AAT was also expressed in the MAP levels of the biomarkers associated with GvHD risk score. At baseline, there was no difference in MAPs between study subjects and controls (median 0.22 vs. 0.23, respectively; FIG. 4A). However, at day 28, patients treated with AAT had reduced MAP scores compared to controls (median 0.10 vs. 0.14, respectively; FIG. 4B). Thus, AAT treatment in high risk patients alters the subsequent development of SR GVHD by clinical as well as laboratory parameters.

### Example 4. AAT for Treating Chronic GvHD

Two patients with chronic GvHD were subjected to compassionate AAT treatment (Glassia®, for intravenous administration). One of the patients, a 46 y.o. male (for whom data is available), developed chronic GvHD following allogenic transplantation after being diagnosed with classical Hodgkin's lymphoma. Several years post transplantation the patient developed severe (stage 4) lung, skin, gut, pharynx and eye conditions associated with chronic GvHD. The patients received weekly AAT doses of 80 mg/KgBW. A significant improvement in the respiratory and nutritional status of the patient have been recorded following 6-month treatment with AAT. Accordingly, the patient was dismissed from hospitalization. Thus, AAT reduces the severity of chronic GVHD and hence increase survival rates of patients with chronic GvHD.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method for reducing the risk to develop graft versus host disease (GvHD) in a subject in need thereof, the method comprising administering to the subject a pharmaceutical composition comprising alpha-1 antitrypsin (AAT).

2. The method according to claim 1, wherein said administering is performed post transplantation.

3. The method according to claim 1, wherein the pharmaceutical composition is administered in combination with at least one additional pharmaceutically active agent.

4. The method according to claim 3, wherein the at least one additional pharmaceutically active agent is selected from the group of: anti-inflammatory agents, immunomodulatory agents, immunosuppressive agents, Janus-associated kinase inhibitors, methotrexate, mTOR inhibitors, proteasome inhibitors, extracorporeal photopheresis, anti-inflammatory monoclonal antibodies, cytotoxic monoclonal antibodies, mesenchymal stem cell-based therapies and cannabis-based therapies.

5. The method according to claim 1, wherein the subject in need thereof is a subject having a high risk of developing GVHD.

6. The method according to claim 5, wherein said GVHD is steroid refractory GVHD.

7. The method according to claim 1, wherein said reducing the risk to develop GVHD comprises preventing the development of GVHD.

8. The method according to claim 1, wherein said reducing the risk to develop GVHD comprises increasing survival.

9. The method according to claim 1, wherein said reducing the risk to develop GVHD comprises reducing the cumulative incidence of non-relapse mortality.

10. The method according to claim 1, wherein said reducing the risk to develop GVHD comprises preventing or reducing the severity of GvHD effects on organs.

11. The method according to claim 1, wherein said reducing the risk to develop GVHD comprises reducing relapse.

12. The method according to claim 1, wherein said reducing the risk to develop GVHD comprises inducing at least partial response.

13. The method according to claim 11, wherein said reducing the risk to develop GVHD comprises inducing complete response.

14. The method according to claim 2, wherein said transplantation is hematopoietic cellular transplantation.

15. The method according to claim 14, wherein said transplantation is bone marrow transplantation.

16. The method according to claim 1, wherein said administering the pharmaceutical composition comprises administering said pharmaceutical composition in a multiple dosage regimen.

17. The method according to claim 1, wherein said administering comprises intravenous administration.

18. The method according to claim 1, wherein said administering comprises oral administration, or administration via inhalation.

19. The method according to claim 2, wherein said administering is performed at least a week post transplantation.

20. The method according to claim 1, wherein said GvHD is acute GvHD.
